(19) United States
(12) Patent Application Publication
(10) Pub. No.: US 2003/0036734 A1
(43) Pub. Date: Feb. 20, 2003

(54) SAFE SYRINGE

(76) Inventor: Chung-Yu Yang, Taipei (TW)

Correspondence Address: BACON & THOMAS, PLLC, 625 SLATERS LANE, FOURTH FLOOR, ALEXANDRIA, VA, 22314

(21) Appl. No.: 09/931,014

(22) Filed: Aug. 17, 2001

(51) Int. Cl. A61M 5/00

(52) U.S. Cl. 604/226; 604/110

(57) ABSTRACT

A safe syringe having been drawn to a negative pressure status by an inner tube inside the plunge, a grip inserted to the top of the plunge that holds against each other with a catch of the syringe; upon completing the injection, the position of the plunge is changed to release the needle from the syringe, and the grip from the plunge for the negative pressure inside the plunge to fast suck the needle into the inner tube of the plunge, thus to be concealed in the plunge to prevent infection by any one due to accidental sting by the needle.

### BACKGROUND OF THE INVENTION

(a) Field of the Invention

The present invention relates to a safe syringe, and more particularly, to one that fast secures its needle of the syringe into the plunge and firmly sucks the needle inside the plunge to avoid infection due to accidental sting.

(b) Description of the Prior Art

Syringes are popularly used in treatment for the patients. Great and continuous efforts are spent in improving design of medical instruments and medical behavior to eliminate possible infection of virus due to improper contact during the practice of medical behavior or disposal of medical refuses. Medical personnel are highly exposed to the danger of infection particularly by sting of syringe due to act of omission. Since the needle of the syringe has contacted the blood of the patient, accidental sting after the injection could cause the medical personnel to get infected. Therefore, attention from the medical instruments has been concentrated on design of a safe syringe to properly conceal the needle after injection to avoid accidental sting.

So far safe syringes generally available in the market have needles to be ejected into the syringe by a coil. However, upon completing the injection, the operator has to execute complex press procedure before releasing the needle to be ejected into the syringe. Exposure to accidental sting remains while executing such press procedure. Even the needle is ejected into the syringe, it is possible for the need to pierce through the syringe.

### SUMMARY OF THE INVENTION

The primary purpose of the present invention is to provide a safe syringe that is capable of fast sucking the needle into an inner tube in the plunge to avoid accidental sting.

Another purpose of the present invention is to provide a safe syringe that secures the needle inside the plunge for the needle to be fast and firmly hidden inside the plunge.

To achieve the purposes, the inner tube inside the plunge is used to draw the syringe into a status of negative pressure, a grip that holds against to each other with a catch of the needle is provided at the top of the plunge. As a result, upon completing the injection by pushing the plunge down to the bottom of the syringe, the grip at the top of the plunge connects the catch of the needle, and by applying a simple action to change the position of the plunge, the needle is released from the syringe, and the grip from the plunge to fast suck the needle into the inner tube of the plunge by taking advantage of the negative pressure inside the inner tube of the plunge.

### BRIEF DESCRIPTION OF THE DRAWINGS

FIG. 1 is a view showing the appearance of a safe syringe of the present invention.

FIG. 2 is an exploded view of a first preferred embodiment of the present invention.

FIG. 3 is a sectional view of the first preferred embodiment of the present invention.

FIG. 4 is a schematic view showing a grip is engaged to a catch of the first preferred embodiment of the present invention.

FIG. 5 is a sectional view showing that the grip is engaged to the catch of the first preferred embodiment of the present invention.

FIG. 6 is a sectional view showing that the grip is holding against a needle of the first preferred embodiment of the present invention.

FIG. 7 is a schematic view showing that the syringe needle is disengaged from a catch holder provided at top of the syringe of the first preferred embodiment of the present invention.

FIG. 8 is a schematic view showing that the grip is getting loose from the needle of the first preferred embodiment of the present invention.

FIG. 9 is a schematic view showing that the needle has been completely retracted into a plunge of the first preferred embodiment of the present invention.

FIG. 10 is a sectional view showing a second preferred embodiment of the present invention.

FIG. 11 is a schematic view showing that a grip is engaged to a catch in the second preferred embodiment of the present invention.

FIG. 12 is a schematic view showing that the grip and a needle are simultaneously disengaged from a plunge and a syringe of the second preferred embodiment of the present invention.

FIG. 13 is a schematic view showing that the need of the second preferred embodiment of the present invention has been completed retracted into the plunge.

### DETAILED DESCRIPTION OF THE PREFERRED EMBODIMENTS

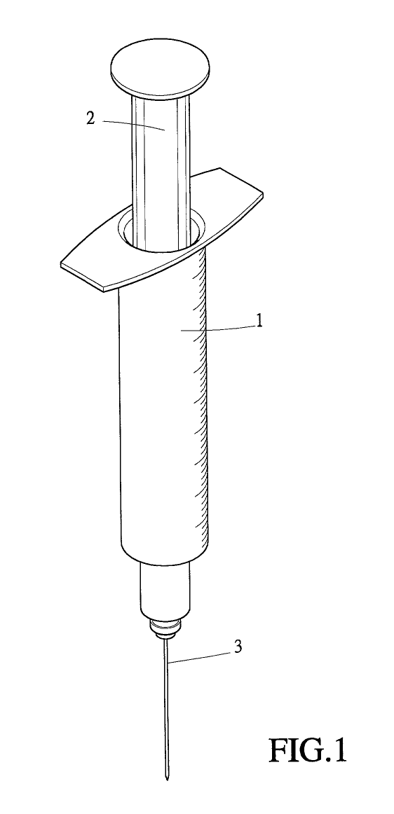

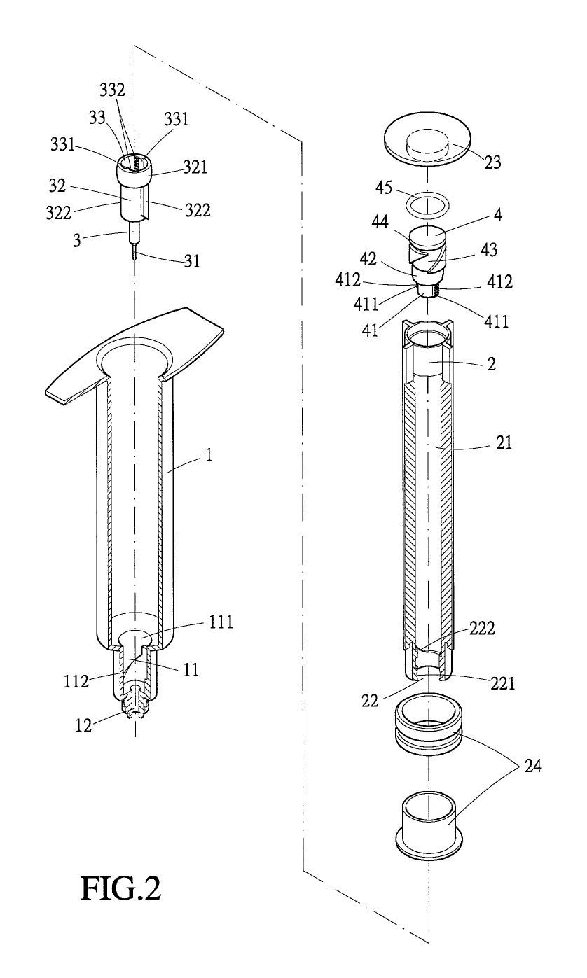

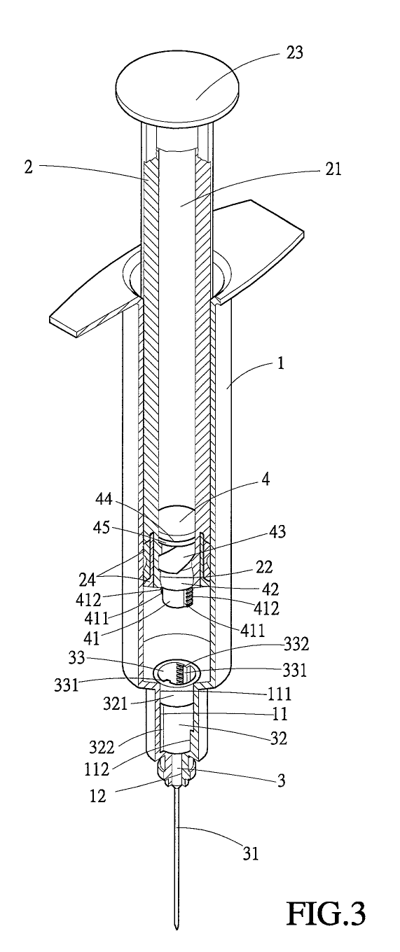

Referring to FIG. 1, the present invention is essentially comprised of a syringe (1) having proper capacity to contain contents from an ampoule, a needle (3) provided at the tip of the syringe (1) to deliver the injection contents, and a plunge (2) provided at the other end of the syringe (1) to push the injection contents through the needle (1). As illustrated in FIGS. 2 and 3, a first preferred embodiment of the present invention has at the tip of the syringe (1) a hole (12) and a catch holder (11); on the needle (3), a catch (32) to be inserted into the catch holder (11) is formed at the rear end of the needle tip (31). The needle (3) is loaded into the syringe (1) from the end where the syringe (1) receives insertion of a plunge (2), then penetrates the syringe (1) through the hole (12) while leaving the catch (32) into the catch holder (11).

A first engagement area (111) and a first engagement section (321) as well as a first slope push area (112) and a first slope push section (322) are respectively and appropriately provided at where the catch holder (11) receives the catch (32). Within, the syringe (1) is firmly received by the catch (32) due to tight engagement between the first engagement section (321) and the first engagement area (111) of the catch holder (11). As the catch (32) turns, the first slop push section (322) pushes against the first slope push area (11) of the catch holder (11), in turn, the first engagement section (321) of the catch (32) retreats from the first engagement area (111) of the catch holder (11) to disengage the syringe (3) from the syringe (1).

Furthermore, said plunge (2) contains an inner tube (21) pre-drawn to a negative pressure status. The front end of the inner tube (21) is provided with an opening (22) to receive a grip (4) and the other end is provided with a cover (23) to close up the inner tube (21). Said grip (4) is inserted into the inner tube (23) from the end where the plunge (2) receives the insertion by the cover (23), and closes up the opening (22) of the plunge (2). Once the inner tube (21) is drawn to the negative pressure status, the other end of the inner tube (21) is closed by the cover (23) to maintain such negative pressure status inside the inner tube (21) for the entire plunge (2). Both of said opening (22) and the grip (4) are respectively provided at where appropriately a second engagement area (221) and a second engagement section (42) as well as a second slope push area (222) and a second slope push section (43). Said grip (4) is firmly engaged with the plunge (2) by tight engagement between the second engagement section (42) and the second engagement area (221) of the opening (22) as well as the restriction by the first slope push section (43) and the second slope push area (222), thus to prevent the grip (4) from being sucked into the inner tube (21) of the plunge (2). Furthermore, once the plunge (2) turns against the grip (4), the secondary engagement section (43) retreats from the second engagement area (221) of the opening (22) to release the grip (4) from the plunge (2). The grip (4) is freed from restriction between it and the opening (22) as the grip (4) rotates to disengage its second slope push section (43) from the second slop push area (222) of the opening (22), and said grip (4) is immediately sucked into the bottom of the inner tube (21) due to the negative pressure present in the inner tube (21).

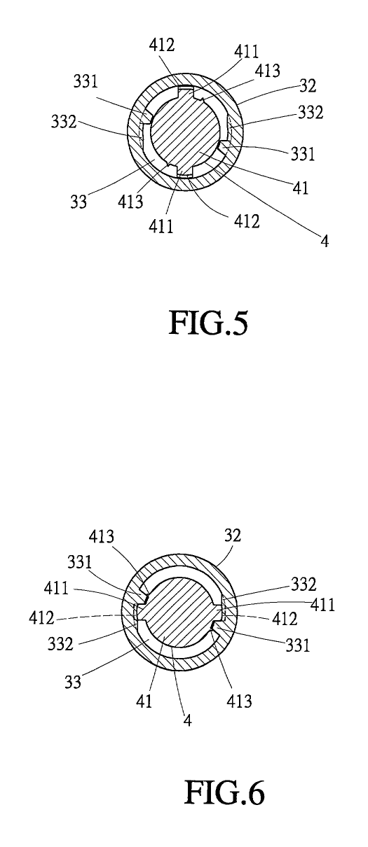

An insertion (41) of the grip (4) protruding from the front end of the plunge (2) passes through an insertion hole (33) formed at the edge of the catch (32) of the needle (3). A first stopper (331) and a second stopper (411) holding against each other are respectively provided on the inner circumference of the insertion hole (33) and on the outer circumference of the insertion (41). A first retainer (332) and a second retainer (412) are provided at where both of said first and second stoppers (331, 411) holding against each other. As illustrated in FIGS. 5 and 6, when said insertion (41) passes through the insertion hole (33) and rotates, both said second and first stopper (411, 331) between the insertion (41) and the insertion hole (33) hold against each other while both said first and second retainers (332, 412) are buckled to each other for the grip (4) to retain the catch (32). Said catch (32) turns along with the grip (4).

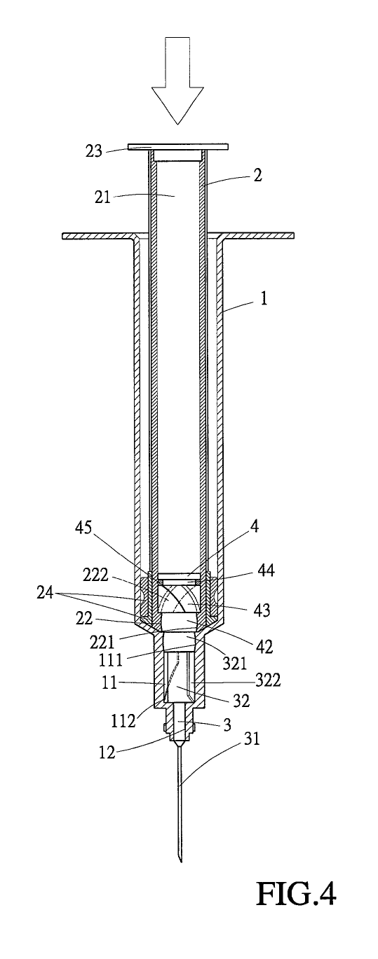

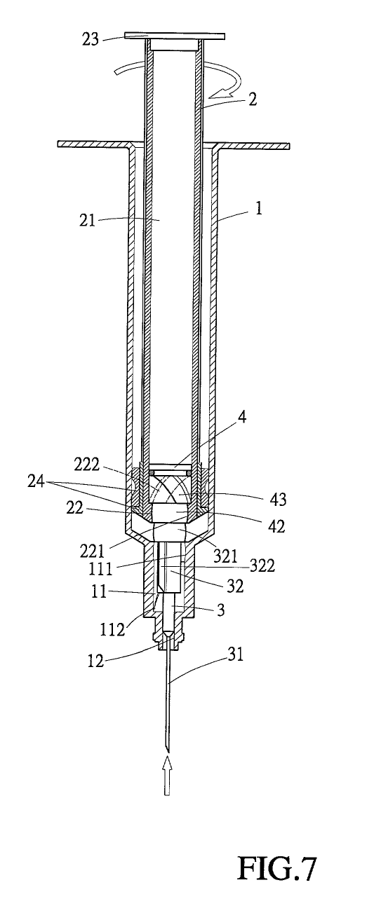

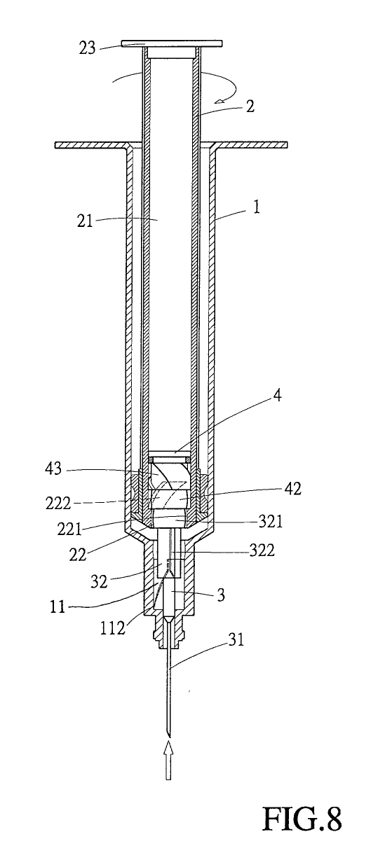

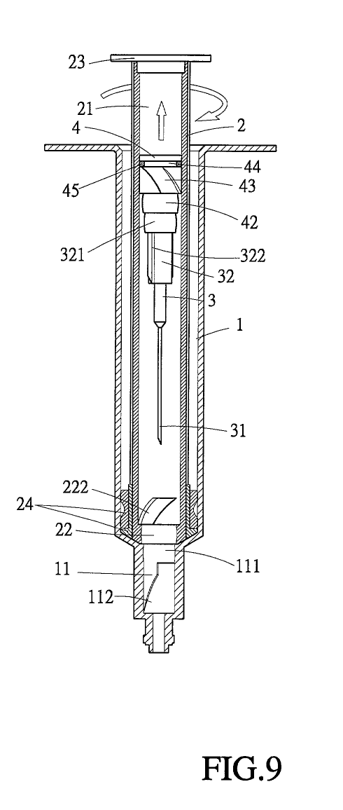

Now referring to FIGS. 4 and 5, upon completing the injection, the plunge (2) is completely pushed down to the bottom of the syringe (1), and the insertion (41) of the grip (4) at the front end of the plunge (2) is inserted in the insertion hole (33) in the catch (32) of the needle (3). As illustrated in FIGS. 6 and 7, by turning the plunge (2), the catch (32) of the needle (3) is retained by the grip (4) and the catch (32) of the needle (3) turns along with the grip (4). When the plunge (2) continues to be turned, the first slope push section (322) of the catch (32) pushes against the first slope push area (112) of the catch holder (11) for the first engagement section (321) on the catch (32) of the needle (3) to retreat from the first engagement area (111) on the catch holder (11) of the syringe (1), and for the needle (3) to disengage from the syringe (1). As illustrated in FIG. 8, once the catch (32) of the needle (3) completely retreats from catch holder (11) of the syringe (1), the needle (3) is prevented from turning as stopped by the root of the first slope push area (112) of the syringe (1). Meanwhile, as the plunge (2) continue turning, the grip (4) turns against the plunge (2), the second engagement section (43) of the grip (4) retreats from the second engagement area (221) of the opening (22) when pushed by the second slope push area (222) of the opening (22) and the second slope push section (43) of the grip (4), thus to release the grip (4) from the plunge (2) As illustrated in FIG. 9, once the second slope push section (43) is disengaged from the second slope push area (222) of the opening (22) as the grip turns, the grip is no longer restricted by the opening (22), the grip is immediately sucked into the bottom of the inner tube (21) by the negative pressure present in the inner tube (21). Since the needle (3) is retained by the grip (4), it is sucked into the inner tube (21) of the plunge (2) at the same time for the needle tip (31) to be fully concealed inside the plunge (2) to avoid infection due to accidental sting.

As illustrated in FIGS. 2 and 8, a piston (24) is inserted to the peripheral of the front end of the plunge (2) to improve tightness of the syringe (1) without causing leakage of the injection contents. Also, a circular groove (44) inserted with leak-proof ring (45) is formed on the outer circumference of the grip (4) to improve air-tightness of the grip (4) and the inner tube (21) as the leak-proof ring (45) is located between the grip (4) and the inner tube (21) of the plunge (2). In turn, the grip faithfully operates by being subject to the suction of the negative pressure, and the grip (4) is firmly secured at the bottom of the inner tube (21) to hold steady the needle (3) inside the plunge (2).

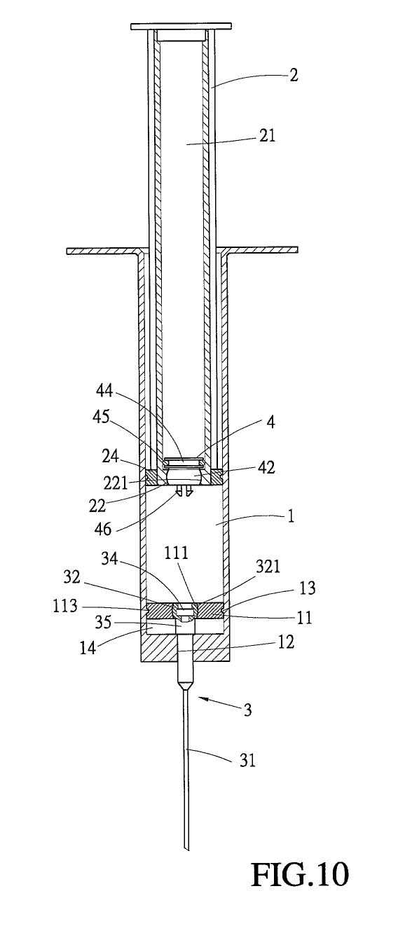

Furthermore, as illustrated in FIG. 10, in a second preferred embodiment of the present invention, a hole (12) to allow passage of the needle (3) is provided at the front end of the syringe (1), a catch holder (11) for the needle (3) to engage the syringe (1) is separately provided inside the syringe (1), a catch (32) is provided on the other end of the needle tip (32) for the needle (3), and a first engagement area (111) and a matching first engagement section (321) are respectively provided on the catch holder (11) and the catch (32); and a first positioning area (13) and a matching second positioning area (113) are respectively provided at where the syringe (1) engages the catch holder (11). The entire needle (3) is positioned at the front end of the syringe (1) as the catch (32) is tightly engaged with the catch holder (11) and the second positioning area (113) of the catch holder (11) is retained by the first positioning area (13) of the syringe (1). A space to accommodate the injection contents is provided in the rear of the catch holder (11) for the syringe (1) while the catch holder (11) remains fixed in position as retained by the first and the second positioning areas (13, 113). A stop section (35) formed for the needle (3) between the needle tip (31) and the catch (32) helps hold steady the needle (3) to prevent it from being moved as the plunge (2) pushes. Relatively, another space (14) is formed between the catch holder (11) and the bottom of the syringe (1) to allow the catch holder (11) retreat when pushed by the plunge (2).

An opening (22) is formed at the front end of the inner tube (21) for the plunge (2), and a piston (24) to push the injection contents is provided on the outer circumference of the plunge (2). The grip (4) is directly inserted to the opening (22) of the plunge (22) and a second engagement area (221) and a matching second engagement section (42) are respectively provided at where the opening (22) is engaged to the grip (4). Similarly, a circular groove (44) inserted with a leak-proof ring (45) can be provided on the outer circumference of the grip (4) with the leak-proof ring (45) inserted between the grip (4) and the inner tube (21) of the plunge (2) thus to improve the air-tightness between the grip (4) and the inner tube (21). Hook (46) and latching hole (34) are respectively formed on the end of the catch (32) of the needle (3) to secure the holding between the grip (4) and the needle (3) when the hook (46) and the latching hole (34) are engaged.

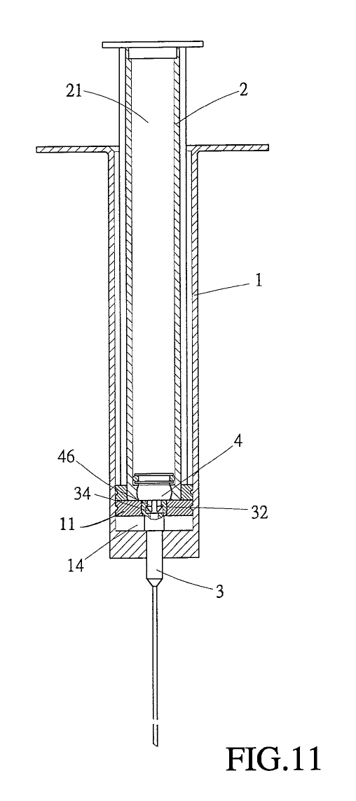

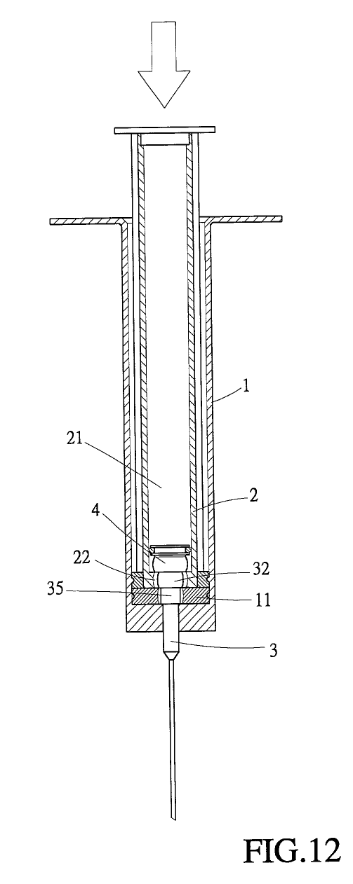

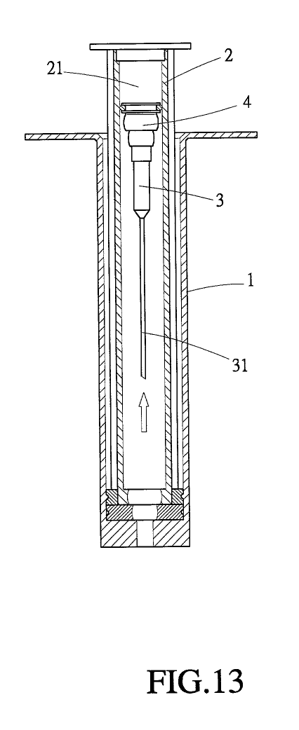

Accordingly, upon completing the injection as the plunge (2) pushes into to the syringe (1), the plunge (2) is fully pushed down to the bottom of the syringe (1) as illustrated in FIG. 11. Wherein, the hook (46) of the grip (4) at the front end of the plunge (2) penetrates into the latching hole (34) in the catch (32) of the needle (3) for form a firm holding force from the grip (4) against the needle (3). As the plunge (2) continues to move down to the syringe (1), the catch (11) is pushed into the retreat section (14). As illustrated in FIG. 12, while the needle (3) is prevented from moving due to the stop section (35), the catch (32) is disengaged from the catch holder (11) thus to release the needle (3) from the syringe (1). Meanwhile, the grip (4) at the front end of the plunge (2) is pushed by the needle (3) and retreats into the inner tube (21) of the plunge (2), thus to release the grip (4) from the opening (22) of the plunge (2). As illustrated in FIG. 13, the grip (4) is sucked into the inner tube (21) due to the negative pressure present in the inner tube (21) of the plunge (2). The needle (3) for being held by the grip (4) is also sucked into the inner tube (21) of the plunge (2) with the needle tip (31) fully concealed inside the plunge (2) to effectively avoid infection due to accidental sting by the needle tip (31).

As disclosed above, the present invention by having drawn the plunge inner tube into a status of negative pressure, the grip at the front end of the plunge to retain the catch of the needle, turning the plunge to release the needle from the syringe and the grip from the plunge, and immediately sucking the needle into the plunge inner tube through the negative pressure present inside the inner tube to firmly secure the needle inside the plunge and the needle tip in the plunge to effectively avoid infection due to accidental sting by the needle tip. A patent is duly filed accordingly.

I claim,

1. A safe syringe comprised of a syringe having a proper capacity to accommodate injection contents; a needle at the front end of the syringe to inject the contents, a plunge inserted into one end of the syringe to push the contents towards the needle; wherein, a hole and a catch holder are provided on the front end of the syringe, and a catch to engage the catch holder is provided at the rear end of a needle tip for the needle so to accommodate the needle inside the syringe to facilitate the needle tip to penetrate through the syringe; characterized by that:
said inner tube of the plunge having been drawn to a negative pressure status, a grip retained by the needle inserted in the front end of the plunge for the grip at the front end of the plunge to engage with the catch of the needle upon completing the injection when the plunge being pushed down to the bottom of the syringe, then by simply changing the position of the plunge, the needle and the grip respectively disengaged from the syringe and the plunge for the needle to be fast sucked into the inner tube due to presence of negative pressure in the inner tube of the plunge to fully conceal the needle inside the plunge.

2. A safe syringe as claimed in claim 1, wherein, a first engagement area and a first engagement section, and a first slope push area and a first slope push section are provided at where said catch is inserted into the catch holder to firmly engage the catch to the syringe as the first engagement section being close engaged with the first engagement area of the catch; as the catch turns, the first slope push section pushes against the first slope push area for the first engagement section of the catch to retreat the first engagement area of the catch thus to disengage the needle from the syringe; an opening provided at the frond end of the inner tube of the plunge to receive the grip with the other end provided a cover to close the inner tube; said grip being inserted into the inner tube from the end having the cover to close up the opening; the other end of the inner tube being closed to maintain its negative pressure status inside by the cover once the entire plunge being drawn to negative pressure in the inner tube; a second engagement area and a second engagement section, and a second slope push area and a second slope push section respectively provided at where the grip engages the opening for the grip to firmly engage with the plunge and to prevent the grip from being sucked into the inner tube of the plunge as restricted by the engaged second slope push area and section and the tight engagement of the second engagement area and section; the second slope push section of the grip pushing against the second slope push area of the opening as the plunge turns against the grip for the second engagement section of the grip to retreat from the second engagement area of the opening to release the grip from the plunge; the grip continues to turn to disengage its second slope push section from the second slope push area of the opening, thus to free the grip from the opening and immediately sucked into the bottom of the inner tube due to the negative pressure present in the inner tube.

3. A safe syringe as claimed in claim 2, wherein, an insertion of the grip protrudes from the frond end of the plunge, an insertion hole formed at the edge of the catch of the needle to receive the insertion, a first stopper and a second stopper holding against each other respectively provided in the inner circumference of the insertion hole and the outer circumference of the insertion; a first retainer and a second retainer respectively provided at where corresponding to said first and second stoppers holding against to each other; the insertion passing through the insertion hole rotates for the first and the second stopper to hold against each other between the insertion and the insertion hole while the first retainer engages the second retainer for the grip to hold against the catch and for the catch to turn along with the grip.

4. A safe syringe as claimed in claim 3, wherein, a stopper latch is provided for the grip by the side of the second stopper of the insertion at an spacing equal to the width of the first stopper so that once both stopper hold against each other, the stopper latch holds against the other side of the first stopper to hold steady both stoppers, thus to even firmly secure the grip to the needle.

5. A safe syringe as claimed in claim 1, wherein, said catch holder is inserted to the front end inside the plunge with the first engagement area and the first engagement section closed engaged to each other respectively provided on said catch holder and said catch; a first positioning area and a second positioning area respectively provided at where said plunge inserted into the catch holder to position the entire needle at the front end of the syringe as restricted by the engaged first and second positioning areas of the catch holder and syringe and the catch holder being close engaged with the catch; a space formed in the rear of the catch holder for the syringe to accommodate the injection contents while said catch holder prevents from movement as restricted by the first and the second positioning areas; a stop section formed between the needle tip and the catch holder for the needle to firmly secure the needle in position without being affected by the push of the plunge; another space provided between the catch holder and the bottom of the syringe for the catch holder to retreat when subject to push by the plunge; an opening formed at front end of the inner tube of the plunge to receive direct insertion by the grip; a second engagement area and a second engagement section close engaged to each other being respectively provided at where the opening engaged with the grip; the plunge being completely pushed down to the bottom of the syringe upon completing injection for the grip to firmly hold the syringe; the catch holder being pushed into the retreat section as the plunge continues to push down the syringe; the needle remains steady as being restricted by the stopper section while relatively the catch being disengaged from the catch holder to release the needle from the syringe, and the grip at the front end of the plunge retreats to the plunge inner tube as being pushed by the needle to release the opening of the plunge from the grip for both of grip and needle to be sucked into the plunge inner tube due to negative pressure present therein.

6. A safe syringe as claimed in claim 5, wherein, hook and latching hole are respective provided on the front end of the grip and the end of the catch of the needle to firmly secure the grip and the needle as the hook is engaged in the latching hole.

7. A safe syringe as claimed in claim 1, wherein, a groove inserted with leak-proof ring is provided on the outer circumference of the grip and located between the grip and the plunge inner tube to improve air tightness between the grip and the inner tube for the grip to operate being subject to suction by negative pressure to firmly hold against the bottom of the inner tube, thus to secure the needle inside the plunge.

8. A safe syringe as claimed in claim 1, wherein, a piston is inserted on the outer circumference at the front end of the plunge to improve the air tightness between the plunge and the syringe.
